(19) United States
(12) Patent Application Publication
(10) Pub. No.: US 2023/0409999 A1
(43) Pub. Date: Dec. 21, 2023

(54) CONTROL DEVICE, CONTROL SYSTEM, AND CONTROL METHOD

(71) Applicant: OLYMPUS CORPORATION, Tokyo (JP)

(72) Inventor: Masato NARUSE, Tokyo (JP)

(73) Assignee: OLYMPUS CORPORATION, Tokyo (JP)

(21) Appl. No.: 18/242,701

(22) Filed: Sep. 6, 2023

Related U.S. Application Data
(63) Continuation of application No. PCT/JP2021/020024, filed on May 26, 2021.

(51) Int. Cl. G06Q 10/0631; G16H 40/20

(52) U.S. Cl. CPC G06Q 10/063112; G16H 40/20

(57) ABSTRACT

Provided is a control device for controlling a schedule of at least one medical event that includes at least one of a surgery, an examination, and a medical interview. The control device includes at least one processor that includes hardware. The at least one processor is configured to: grasp progress of the medical event that is currently being performed; and determine whether adjustment of the schedule is necessary or not, based on the progress. The schedule includes information about a required time of each stage that is performed in each medical event. The at least one processor is configured to: grasp a current stage of the medical event that is currently being performed, based on an endoscopic image of the medical event that is currently being performed; and grasp the progress based on the current stage and the schedule.

### CROSS-REFERENCE TO RELATED APPLICATIONS

This is a continuation of International Application PCT/JP2021/020024 which is hereby incorporated by reference herein in its entirety.

### TECHNICAL FIELD

The present invention relates to a control device, a control system, and a control method that control a schedule for a medical event in a hospital.

### BACKGROUND ART

Generally, in a hospital, a schedule is created in advance for a plurality of surgeries that are to be performed in a day, based on patients, operative procedures, operating rooms, equipment, and medical staff such as doctors, nurses and engineers. When a surgery is started, the surgery is possibly not performed as scheduled and may be prolonged. If a surgery is prolonged, additional medical staff or equipment may become necessary for the surgery, or a plan for another surgery may have to be adjusted. To swiftly grasp a delay in a surgery, a staff member outside the operating room regularly checks progress of the surgery by calling the operating room or by taking a look inside the operating room. Such checking of the progress of a surgery is burdensome to the staff member. Accordingly, there is proposed a technique of automatically grasping the progress of a surgery (for example, see PTL 1).

PTL 1 discloses detection of progress of a surgery based on on/off of a plurality of machines used in the surgery, and real-time notification of the progress to outside of an operating room. According to PTL 1, in a case where a surgery is taking longer than is scheduled, a staff member outside the operating room is notified in real-time by the delay in the surgery being displayed on a display.

### CITATION LIST

### Patent Literature

### {PTL 1}

Japanese Unexamined Patent Application, Publication No. 2014-184126

### SUMMARY OF INVENTION

An aspect of the present invention is a control device for controlling a schedule of at least one medical event that includes at least one of a surgery, an examination, and a medical interview, the control device including at least one processor that includes hardware, wherein the at least one processor is configured to: grasp progress of the medical event that is currently being performed; and determine whether adjustment of the schedule is necessary or not, based on the progress, the schedule includes information about a required time of each stage that is performed in each medical event, and the at least one processor is configured to: grasp a current stage of the medical event that is currently being performed, based on an endoscopic image of the medical event that is currently being performed; and grasp the progress based on the current stage and the schedule.

Another aspect of the present invention is a control system for controlling a schedule of at least one medical event that includes at least one of a surgery, an examination, and a medical interview, the control system including: a server that stores the schedule; and a control device that is connected to the server via a communication network, wherein the control device includes at least one processor that includes hardware, the at least one processor is configured to: grasp progress of the medical event that is currently being performed; and determine whether adjustment of the schedule is necessary or not, based on the progress, the schedule includes information about a required time of each stage that is performed in each medical event, and the at least one processor is configured to: grasp a current stage of the medical event that is currently being performed, based on an endoscopic image of the medical event that is currently being performed; and grasp the progress based on the current stage and the schedule.

Another aspect of the present invention is a control method for controlling a schedule of at least one medical event that includes at least one of a surgery, an examination, and a medical interview, the control method including: grasping progress of the medical event that is currently being performed; and determining whether adjustment of the schedule is necessary or not, based on the progress, wherein the schedule includes information about a required time of each stage that is performed in each medical event, and the grasping includes: grasping a current stage of the medical event that is currently being performed, based on an endoscopic image of the medical event that is currently being performed; and grasping the progress based on the current stage and the schedule.

### BRIEF DESCRIPTION OF DRAWINGS

FIG. 1 is a block diagram showing a configuration of a control device and a control system according to an embodiment of the present invention.

FIG. 2 is a diagram showing an example of a schedule for surgeries.

FIG. 3 is a flowchart of a control method for the schedule according to the embodiment of the present invention.

FIG. 4 is a flowchart of a progress grasping routine for a surgery in the flowchart in FIG. 3.

FIG. 5 is a flowchart of a reasonability check routine for a proposed adjustment in the flowchart in FIG. 3.

FIG. 6 is a diagram for describing an example of a method of inputting a proposed adjustment for the schedule.

FIG. 7 is a diagram for describing an example of a method of selecting a concerned person who is to be notified of a change in the schedule.

### DESCRIPTION OF EMBODIMENTS

Hereinafter, a control device, a control system, and a control method according to an embodiment of the present invention will be described with reference to the drawings.

A control device 10 and a control system 100 according to the present embodiment are for controlling a schedule of at least one medical event that is performed in a hospital. A medical event includes at least one of a surgery, an examination, and a medical interview. In the present embodiment, a schedule of a surgery will be described.

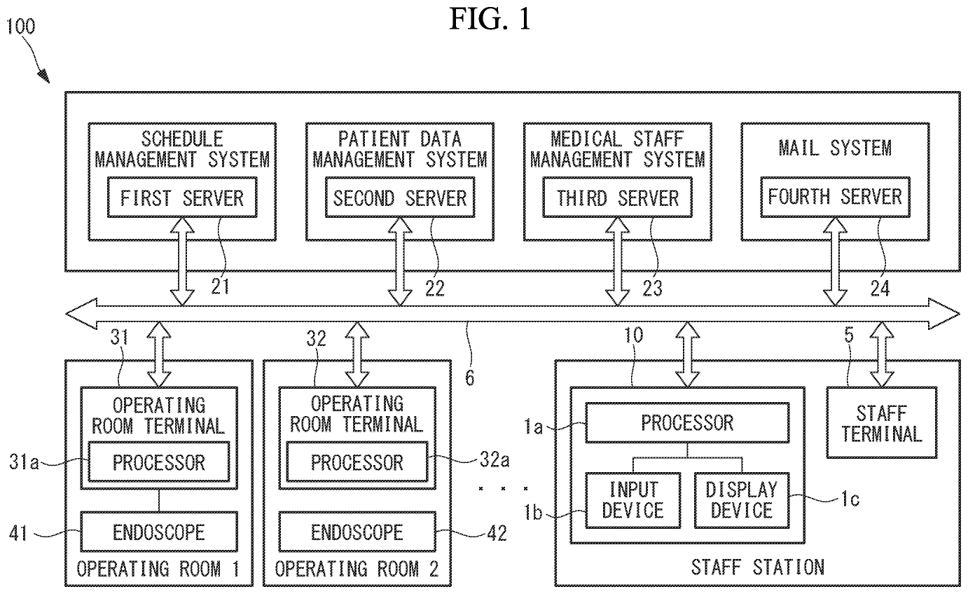

As shown in FIG. 1, the control system 100 includes a plurality of servers 21, 22, 23, 24 that store information and data in a hospital, a plurality of operating room terminals 31, 32, . . . , endoscopes (image pickup devices) 41, 42, . . . that acquire endoscopic images in a surgery, a staff terminal 5 that is disposed outside an operating room, the control device 10, and a communication network 6.

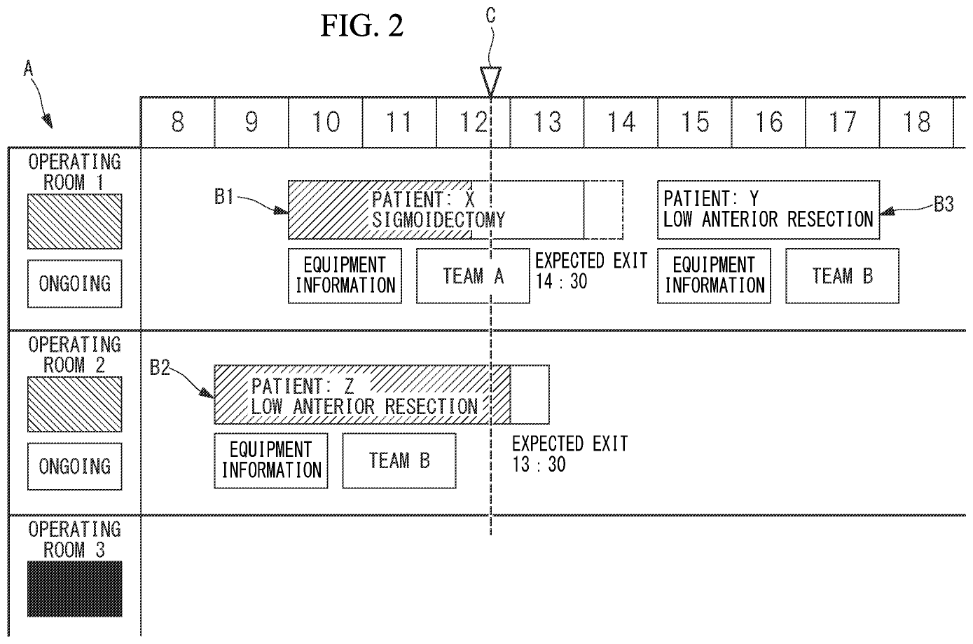

The first server 21 configures a schedule management system, and as shown in FIG. 2, the first server 21 stores a schedule A of at least one surgery B1, B2, B3, . . . performed in the hospital, the schedule A being created in advance by staff. The schedule A includes an advance plan for each surgery B1, B2, B3 set in advance, including a start time, an end time, an operating room, equipment to be used, and assigned medical staff. In FIG. 2, an arrow C indicates a current time, and the surgeries B1, B2 are currently being performed.

Each surgery B1, B2, B3, . . . includes at least one stage including section, organ resection, enucleation, or suturing, for example. In the schedule A, an individual schedule of each surgery B1, B2, B3, . . . including a required time of each stage from start of the surgery is set, for example.

The second server 22 configures a patient data management system, and stores personal information, a diagnosis result and the like of a patient.

The third server 23 configures a medical staff management system, and stores personal information, work shift information and the like of a medical staff member. Medical staff includes doctors, nurses, nursing assistants, engineers, and the like.

The fourth server 24 configures a mail system, and assigns an email address to each medical staff member, each clerical worker, and the like, and transmits/receives emails.

The operating room terminal 31, 32, . . . is a personal computer, for example, and includes a processor 31a, 32a, . . . including hardware such as a central processing unit (CPU). The operating room terminals 31, 32, . . . and the endoscopes 41, 42, . . . are disposed in a plurality of operating rooms (medical rooms) 1, 2, . . . where surgeries are performed.

The staff terminal 5 is used by a staff member outside the operating rooms 1, 2, . . . , and is disposed in a staff station installed outside the plurality of operating rooms 1, 2, . . . , for example.

The control device 10 is any computer such as a personal computer, and is installed in the staff station, for example. The control device 10 includes a processor 1a including hardware such as a CPU, an input device 1b, and a display device 1c. The input device 1b includes freely selected input devices such as a mouse, a keyboard, and a touch panel. The display device 1c includes a freely selected display such as a liquid crystal display.

The control device 10 may also be installed at any location in the hospital outside the staff station, and may be installed in any of the operating rooms 1, 2, . . . , for example. Furthermore, any device that is installed in the hospital and that includes a processor may be used as the control device 10. For example, one of the operating room terminals 31, 32, . . . , or one of the servers 21, 22, 23, 24 may be used as the control device 10.

The communication network 6 is a local area network (LAN) that is installed in the hospital, for example. The communication network 6 may be a communication network of a type other than the LAN. The control device 10, the servers 21, 22, 23, 24, the terminals 31, 32, . . . , 5, and the endoscopes 41, 42, . . . are connected to the communication network 6, and are capable of communicating with one another via the communication network 6.

The control device 10 acquires information and data necessary to control the schedule A from the servers 21, 22, 23, 24, the terminals 31, 32, . . . , 5, and the endoscopes 41, 42, . . . via the communication network 6. The control device 10 grasps progress of surgeries currently being performed in the operating rooms 1, 2, . . . in real-time, and controls the schedule A based on the progress.

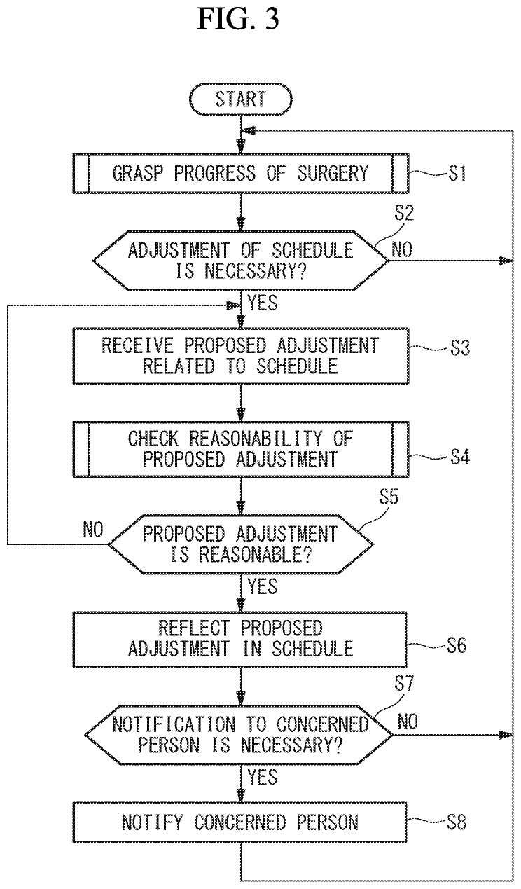

Next, a control method for schedule performed by the control device 10 and the control system 100 will be described with reference to FIGS. 3 to 5.

The processor 1a of the control device 10 performs the control method shown in FIGS. 3 to 5. The control method includes step S1 of grasping progress of each surgery currently being performed, step S2 of determining whether adjustment of the schedule A is necessary or not based on the progress, step S3 of receiving a proposed adjustment related to the schedule A, step S4 of checking reasonability of the proposed adjustment, step S5 of determining whether the proposed adjustment is reasonable or not based on a check result, step S6 of reflecting the proposed adjustment in the schedule A, step S7 of determining whether the notification of change in the schedule A to a concerned person is necessary or not, and step S8 of notifying the concerned person of the change in the schedule A.

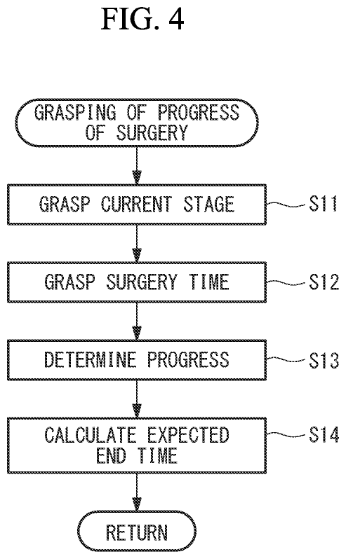

In step S1, the processor 1a acquires in real-time progress-related information related to progress of the surgeries B1, B2 currently being performed from the devices 31, 32, 41, 42 in the operating rooms 1, 2, grasps progress of each surgery B1, B2 based on the progress-related information, and calculates an expected end time of each surgery B1, B2 based on the progress. More specifically, step S1 includes steps S11 to S14 as shown in FIG. 4.

In step S11, the processor 1a receives an endoscopic image of the surgery B1 as the progress-related information from the endoscope 41, and grasps the current stage of the surgery B1 by analyzing the endoscopic image. For example, properties of an endoscopic image of each stage are stored in advance in a storage device (not shown) of the control device and the current stage is grasped by comparing the properties of the endoscopic image that is received with the properties that are stored. The processor 1a may also grasp the current stage by grasping properties of an endoscopic image by which switching of stages can be determined (for example, display of a specific organ, or start of use of a specific instrument). The processor 1a also grasps the current stage of the surgery B2 by receiving an endoscopic image of the surgery B2 from the endoscope 42.

A surgery log is recorded in the operating room terminal 31, 32 when a medical staff member in the operating room 1, 2 inputs progress of the surgery B1, B2 in the operating room terminal 31, 32. The processor 1a may acquire the surgery log that is the progress-related information from the operating room terminal 31, 32, and grasp the current stage of the surgery B1, B2 based on the surgery log.

Next, in step S12, the processor 1a grasps a surgery time of each surgery B1, B2 based on the start time set in the schedule A and the current time. The surgery time is an elapsed time between the surgery start time and the current time. The current time is grasped by referring to a clock in the processor 1a, for example.

Next, in step S13, the processor 1a determines progress of the surgery B1 based on the current stage and the surgery time of the surgery B1, and determines progress of the surgery B2 based on the current stage and the surgery time of the surgery B2. More specifically, the processor 1a determines whether the current stage is as scheduled, earlier than scheduled, or later than scheduled, by comparing the surgery time and a required time for the current stage in the schedule A.

Next, in step S14, the processor 1a calculates an expected end time of each surgery B1, B2 based on the progress determined in step S13. For example, in the case where the surgery is proceeding fast, an expected end time that is earlier than the end time set in the schedule A by a difference between the surgery time and the required time is calculated. In the case where the surgery is proceeding slowly, an expected end time that is later than the end time set in the schedule A by the difference between the surgery time and the required time is calculated.

A predetermined scene of a surgery may be taken as a gate in step S1, and a time that is a predetermined required time after the gate may be calculated as the expected end time.

For example, a scene of enucleation may be recognized based on the endoscopic image, and 30 minutes after scene recognition may be calculated as the expected end time. Alternatively, a scene during the surgery may be finely recognized based on the surgery log of the surgery B1, B2, and the expected end time may be calculated based on the scene that is recognized.

In the case where the surgery is proceeding fast or slowly, the processor 1a may cause the fastness or delay in the surgery to be reflected in the schedule A by adding the expected end time in the schedule A. For example, as shown in FIG. 2, in the case where there is a delay in the surgery B1, extension and the expected end time of the surgery B1 may be displayed by a dotted line. A staff member outside the operating rooms 1, 2 may easily grasp the fastness or delay in the surgery, and the expected end time.

Next, in step S2, the processor 1a compares the expected end time calculated in step S14 with the end time that is set in the schedule A. In the case where the expected end time is the same as the end time that is set, or is earlier than the end time that is set, the processor 1a determines that it is not necessary to change the schedule A.

On the other hand, in the case where the expected end time is later than the end time that is set, the processor 1a determines that adjustment of the schedule A is necessary. In this case, the processor 1a notifies a staff member outside the operating rooms 1, 2, . . . of necessity of rescheduling by causing a notification urging adjustment of the schedule A to be displayed on the display device 1c, for example. The processor 1a may further notify of a reason for determining that adjustment is necessary, namely, the surgery B1, the expected end time of which is later than the end time that is set, for example.

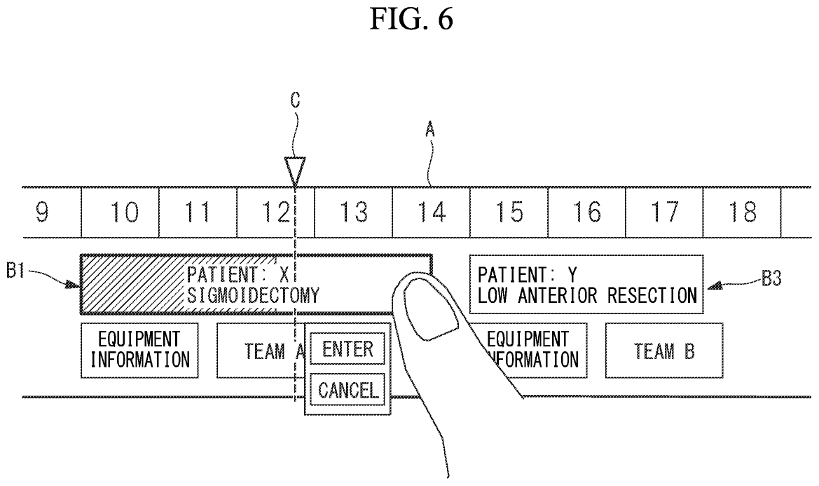

In response to the notification, the staff member inputs adjustment related to an advance plan of at least one surgery to the control device 10 using the input device 1b. For example, as shown in FIG. 6, the staff member inputs adjustment to delay the end time of the surgery B1 with a delay, by performing a touch operation on a touch screen where the schedule A is displayed. After the input, when an enter button is clicked, a proposed adjustment related to the schedule A including the adjustment that is input is transmitted from the input device 1b to the processor 1a.

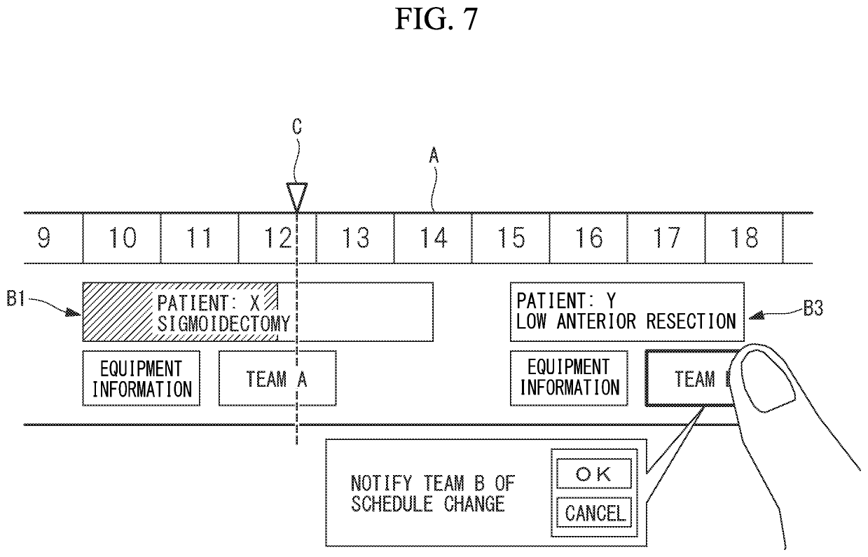

In step S3, the processor 1a receives the proposed adjustment related to the schedule A from the input device 1b. In the example in FIG. 7, adjustment of the end time of the surgery B1, and adjustment of a start time and an end time of the surgery B3 are input. In addition to adjustment of the start time and the end time, the proposed adjustment may further include change of the operating room, addition of equipment and addition of a staff member, and any adjustment related to advance plans of the surgeries B1, B2, B3, . . . set in the schedule A.

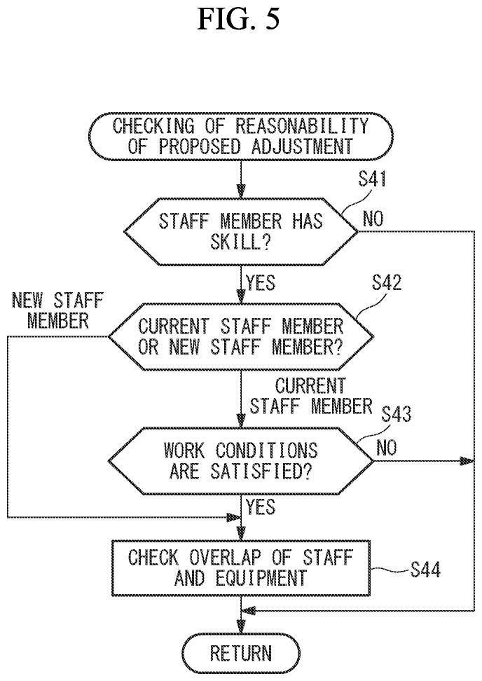

Next, in step S4, the processor 1a checks reasonability of adjustment related to each surgery B1, B2 in the proposed adjustment, in relation to at least one item. More specifically, step S4 includes steps S41 to S44 as shown in FIG. 5.

In step S41, the processor 1a checks whether adjustment related to the surgery B1 is reasonable or not, based on skill of medical staff assigned to the surgery B1 for which there is adjustment.

More specifically, a degree of difficulty of each surgery is stored in the first server 21, and skill information about a skill of each medical staff member, such as medical cases handled in the past, is stored in the third server 23. The processor 1a acquires the level of difficulty of the surgery B1 from the first server 21, and acquires the skill information of a medical staff member assigned to the surgery B1 from the third server 23, and determines whether the medical staff member who is assigned has a skill that is necessary for the surgery B1 or not, based on the level of difficulty of the surgery and the skill information.

In the case where the medical staff member has the necessary skill, the processor 1a determines that adjustment related to the surgery B1 is reasonable, and proceeds to step S42. In contrast, in the case where the medical staff member does not have the necessary skill, the processor 1a determines that adjustment related to the surgery B1 is not reasonable, and ends checking of reasonability of adjustment related to the surgery B1.

The skill that is necessary for a surgery may change during the surgery according to a state of the surgery. Accordingly, the processor 1a may determine the current level of difficulty of the surgery. For example, in the case where the number of times of putting in or taking out a pack from inside the body of a patient or the number of times of replacing a treatment instrument becomes greater than a regular number of times during the surgery, the level of difficulty of the surgery may be determined to have increased than at the start of the surgery, and the skill that is necessary for the surgery may be changed to a higher level.

Steps S42, S43 are performed for the surgery B1 that is currently being performed.

In step S42, the processor 1a determines whether a medical staff member who is assigned is a staff member who is to newly join the surgery B1 or a current medical staff member who is currently taking part in the surgery B1. The determination is performed by comparing the medical staff set in the schedule A with the medical staff set in the proposed adjustment, for example. The processor 1a proceeds to step S43 in the case where a current medical staff member is determined, and proceeds to step S44 in the case where a new medical staff member is determined.

In step S43, the processor 1a checks whether adjustment related to the surgery B1 is reasonable or not, based on work conditions of current medical staff. More specifically, work conditions of each medical staff member are stored in the third server 23. The work conditions include a work start time, a work end time, a criterion for overtime, and the criterion for overtime includes allowable overtime hours. The processor 1a acquires the work conditions of the current medical staff from the third server 23, and checks whether work hours of the medical staff in the proposed adjustment satisfy the work conditions or not. For example, in the case where one overtime hour per day is allowed, the work conditions are determined to be satisfied in the case where the surgery B1 ends before the overtime hour exceeds one hour, and in other cases, the work conditions are determined not to be satisfied.

In the case where the work conditions are satisfied, the processor 1a determines that the adjustment related to the surgery B1 is reasonable, and proceeds to step S44. In contrast, in the case where the work conditions are not satisfied, the processor 1a determines that the adjustment related to the surgery B1 is not reasonable, and ends checking of reasonability of the adjustment related to the surgery B1.

In step S44, the processor 1a checks whether there is a temporal overlap of medical staff and equipment between the surgery B1 and other surgeries B2, B3, . . . . Checking of overlap of medical staff is performed by acquiring the work shift information of the medical staff of each surgery B1, B2, B3 set in the schedule A from the third server 23, and by referring to the work shift information. Checking of overlap of equipment is performed by referring to information about equipment set in the schedule A.

In the case where there is no overlap with respect to both the medical staff and the equipment, the processor 1a determines that the adjustment related to the surgery B1 is reasonable, and in the case where there is overlap with respect to at least one of the medical staff and the equipment, the processor 1a determines that the adjustment related to the surgery B1 is not reasonable. Whether a medical staff member is coming into work or taking a break may also be taken into account in step S44.

The processor 1a also checks reasonability of adjustment related to the surgery B3 according to steps S41 to S44.

Additionally, in step S4, not all of steps S41 to S44 have to be performed, and it suffices if at least one of steps S41, S43, S44 is performed. For example, only step S41 may be performed. In the case where two or more among steps S41, S43, S44 are to be performed, the order of execution can be freely set.

Furthermore, in step S4, all of steps S41 to S44 may be performed regardless of determination results in steps S41, S43, and reasonability may be checked in relation to each of the skill of the medical staff, the work conditions of the medical staff, and overlap of the medical staff and the equipment.

Next, in step S5, in the case where the adjustment related to the surgeries B1, B3 is determined to be reasonable in all of steps S41, S43, S44, the processor 1a determines that the proposed adjustment related to the schedule A is reasonable, and proceeds to step S6.

In step S6, the processor 1a rewrites the schedule A by reflecting each adjustment included in the proposed adjustment in the schedule A that is stored in the first server 21.

Next, in step S7, the processor 1a determines whether it is necessary to notify each concerned person of the surgeries B1, B3 for which there are changes, of the change in the schedule A. The concerned persons are medical staff assigned to the surgeries B1, B3, for example.

For example, a concerned person in the staff station who is close to the control device 10, and concerned persons in the operating rooms 1, 2 who are taking part in the surgeries B1, B2 currently being performed normally grasp a change in the schedule on site. The processor 1a identifies the current location of each concerned person based on the work schedule of each concerned person on the day in question, and determines that notification to a concerned person who is in the staff station or in the operating room 1, 2 is not necessary. Moreover, the processor 1a determines that notification to a concerned person who is not present near the control device 10 nor in the operating rooms 1, 2 is necessary.

In step S7, the processor 1a may determine whether notification is necessary or not, based on information input by the staff to the input device 1b. For example, as shown in FIG. 7, a staff member may select a concerned person to whom a notification should be transmitted, by using the input device 1b, and the processor 1a may determine that notification to the selected concerned person is necessary. In FIG. 7, a team in charge of the surgery B3 is selected, and the notification is transmitted to every concerned person in the team.

Next, in step S8, the processor 1a notifies of the change in the schedule A to only the concerned person for whom notification is determined to be necessary, without notifying of the change in the schedule A to a concerned person for whom notification is determined to be not necessary. For example, the processor 1a transmits, via the fourth server 24, an email notifying of the change in the schedule A to the staff terminal carried by the concerned person.

In the case where it is checked in at least one of steps S41, S43, S44 that the proposed adjustment is not reasonable, the processor 1a determines in step S5 that the proposed adjustment related to the schedule A is not reasonable. In this case, the processor 1a notifies the staff that the proposed adjustment is not reasonable by causing the display device 1c to display a notification asking for another proposed adjustment related to the schedule A, for example, and waits for reception of a next proposed adjustment (step S3). The processor 1a may also transmit a reason for determining that the proposed adjustment is not reasonable to the display device 1c and cause the reason to be displayed.

A staff member responds to the notification displayed on the display device 1c, and inputs another proposed adjustment to the input device 1b and causes the proposed adjustment to be transmitted to the processor 1a.

In the case where a delay occurs in a surgery, additional equipment may become necessary, or an additional nurse who can cope with an emergency may have to be dispatched to the operating room. Accordingly, a staff member outside the operating room has to be able to swiftly grasp the delay in the surgery and necessity of changing plan of the surgery, and to swiftly take necessary measures related to change in the plan. Conventionally, a staff member in the staff station grasps in real-time the progress of all the surgeries being performed by monitoring videos of inside of the plurality of operating rooms 1, 2, . . . , calling the operating rooms 1, 2, . . . , or taking a look inside the operating rooms 1, 2, . . . , for example, and then determines necessity of adjusting the schedule A, creates a proposed adjustment, and checks reasonability of the proposed adjustment. These tasks are burdensome to the staff, and if there is a delay in grasping of the progress, determination of the necessity of adjusting the schedule, and checking of the reasonability of the proposed adjustment, a delay may also be caused in changing of the advance plan of the surgery, and the delay in the surgery is possibly not efficiently handled.

According to the present embodiment, the processor 1a grasps in real-time the progress of each surgery currently being performed, based on the progress-related information acquired from each operating room 1, 2, determines in real-time the necessity of adjusting the schedule A based on the progress, and determines the reasonability of the proposed adjustment related to the schedule A created by the staff. In this manner, a task necessary for rescheduling may be automatically performed without requiring a human, and burden on the staff may be reduced.

Furthermore, when there is a necessity of adjusting the current schedule A, a staff member outside the operating room is notified of the necessity of rescheduling. Accordingly, the staff member outside the operating room may swiftly grasp that there is a necessity of rescheduling based on the notification, and may swiftly take necessary measures, such as inputting of a proposed adjustment related to the schedule A.

Furthermore, when the schedule A is changed, a notification indicating the same is automatically transmitted to a concerned person. Accordingly, the burden on the staff managing the schedule A may be further reduced.

In the embodiment described above, the processor 1a is assumed to perform all of steps S1 to S8, but the processor 1a may instead perform only some of steps S1 to S8.

In a modification, the processor 1a may start the control method described above from step S4. In this case, the tasks in steps S1, S2 are performed by the staff. After receiving the proposed adjustment related to the schedule A in step S3, the processor 1a performs steps S4 to S8. As described above, to determine reasonability of a proposed adjustment, various items such as skill and work conditions of the medical staff have to be considered. Such a task is burdensome to the staff. When the processes from step S4 are automatically performed by the processor 1a, the burden on the staff may be efficiently reduced.

In another modification, the processor 1a may perform only steps S1, S2. In this case, tasks in steps S4 to S8 are performed by the staff. To swiftly cope with the necessity of adjusting the schedule A, such a state has to be swiftly grasped and the necessity of adjustment has to be swiftly determined. Such tasks are burdensome for the staff. When the processes in steps S1, S2 are automatically performed by the processor 1a, the burden on the staff may be efficiently reduced.

In further another modification, the processor 1a does not have to perform steps S7, S8. In this case, tasks in steps S7, S8 are performed by the staff.

In the present embodiment, the control system 100 includes the endoscopes 41, 42 that are in the operating rooms 1, 2, but an image pickup device other than the endoscopes 41, 42 may also be included.

The image pickup device is any device that captures a video of a surgery. For example, the image pickup device may be a camera that is installed in the operating room and that captures a live video of the operating room. In this manner, progress information about a surgery may also be obtained from a video from any type of image pickup device other than the endoscopes 41, 42.

The embodiment described above describes a case of controlling the schedule of a surgery, but a schedule including a medical event other than the surgery may also be controlled. More specifically, the schedule may include at least one of a surgery, an examination, and a medical interview. In the case where the schedule includes an examination, the control system 100 is connected via the communication network 6 to a terminal and an image pickup device in an examination room (a medical room), and in the case where the schedule includes a medical interview, the control system 100 is connected via the communication network 6 to a terminal and an image pickup device in a consultation room (a medical room).

### REFERENCE SIGNS LIST

10 control device
1a processor
21, 22, 23, 24 server
31, 32 operating room terminal
41, 42 endoscope (image pickup device)
5 staff terminal
6 communication network
100 control system
A schedule
B1, B2, B3 surgery (medical event)

1. A control device for controlling a schedule of at least one medical event that includes at least one of a surgery, an examination, and a medical interview, the control device comprising at least one processor that includes hardware, wherein
the at least one processor is configured to:
grasp progress of the medical event that is currently being performed; and
determine whether adjustment of the schedule is necessary or not, based on the progress,
the schedule includes information about a required time of each stage that is performed in each medical event, and
the at least one processor is configured to:
grasp a current stage of the medical event that is currently being performed, based on an endoscopic image of the medical event that is currently being performed; and
grasp the progress based on the current stage and the schedule.

2. The control device according to claim 1, wherein the at least one processor is configured to:
receive a proposed adjustment related to the schedule in a case where adjustment of the schedule is determined to be necessary;
determine whether the proposed adjustment is reasonable or not; and
change the schedule based on the proposed adjustment in a case where the proposed adjustment is determined to be reasonable.

3. The control device according to claim 1, wherein the at least one processor is configured to calculate an expected end time of the medical event that is currently being performed, based on the progress that is grasped.

4. The control device according to claim 3, wherein the at least one processor is configured to determine whether adjustment of the schedule is necessary or not, based on the expected end time and an end time, in the schedule, of the medical event that is currently being performed.

5. The control device according to claim 2, wherein
the schedule includes information about a staff member who is assigned to each medical event, and
the at least one processor is configured to determine whether the proposed adjustment is reasonable or not, based on a skill of the staff member of the medical event for which there is an adjustment in the proposed adjustment.

6. The control device according to claim 2, wherein
the schedule includes information about a staff member who is assigned to each medical event, and
the at least one processor is configured to:
determine whether the staff member of the medical event for which there is an adjustment in the proposed adjustment is a staff member who is to newly take part in the medical event that is currently being performed or a staff member who is currently taking part in the medical event that is currently being performed; and
determine, in a case where it is determined to be the staff member who is currently taking part in the medical event that is currently being performed, whether the proposed adjustment is reasonable or not, based on work conditions of the staff member.

7. The control device according to claim 2, wherein the at least one processor is configured to:
determine whether or not there is temporal overlap of staff and equipment between the medical event for which there is an adjustment in the proposed adjustment and another medical event; and
determine that the proposed adjustment is reasonable in a case where no overlap is determined.

8. The control device according to claim 2, wherein, in a case where the schedule is changed, the at least one processor is configured to notify of a change in the schedule to a concerned person of the medical event that is changed.

9. The control device according to claim 8, wherein the at least one processor is configured to:
determine whether notification of change in the schedule to each concerned person is necessary or not, and determine that the notification is not necessary for at least one of the concerned person who is close to the control device and the concerned person who is taking part in the medical event that is currently being performed; and
not notify of the change in the schedule to the concerned person for whom the notification is determined to be unnecessary.

10. A control system for controlling a schedule of at least one medical event that includes at least one of a surgery, an examination, and a medical interview, the control system comprising:
a server that stores the schedule; and
a control device that is connected to the server via a communication network, wherein
the control device includes at least one processor that includes hardware,
the at least one processor is configured to:
grasp progress of the medical event that is currently being performed; and
determine whether adjustment of the schedule is necessary or not, based on the progress,
the schedule includes information about a required time of each stage that is performed in each medical event, and
the at least one processor is configured to:
grasp a current stage of the medical event that is currently being performed, based on an endoscopic image of the medical event that is currently being performed; and
grasp the progress based on the current stage and the schedule.

11. The control system according to claim 10, further comprising an image pickup device that is disposed in each of at least one medical room where the medical event is performed, wherein
the at least one processor is configured to grasp the progress based on a video of the medical event that is acquired by the image pickup device.

12. A control method for controlling a schedule of at least one medical event that includes at least one of a surgery, an examination, and a medical interview, the control method comprising:
grasping progress of the medical event that is currently being performed; and
determining whether adjustment of the schedule is necessary or not, based on the progress, wherein
the schedule includes information about a required time of each stage that is performed in each medical event, and
the grasping comprises:
grasping a current stage of the medical event that is currently being performed, based on an endoscopic image of the medical event that is currently being performed; and
grasping the progress based on the current stage and the schedule.
